(19) United States
(12) Patent Application Publication
(10) Pub. No.: US 2013/0247918 A1
(43) Pub. Date: Sep. 26, 2013

(54) Auto-Destruct Pre-filled Single-use Syringe

(71) Applicant: Sujoy Kumar Guha, New Delhi (IN)

(72) Inventor: Sujoy Kumar Guha, New Delhi (IN)

(21) Appl. No.: 13/992,210

(22) PCT Filed: Dec. 2, 2011
(86) PCT No.: PCT/IN2011/000821

§ 371 Date: Jun. 6, 2013

(30) Foreign Application Priority Data
Dec. 6, 2010 (IN) 2903/DEL/2010

(51) Int. Cl. A61F 6/02

(52) U.S. Cl. CPC A61F 6/02
USPC 128/842

(57) ABSTRACT

An Auto-Destruct, Pre-loaded Syringe is provided which is suitable for pre-loading with highly viscous, sticky and reactive drug and for withstanding higher pressure required for loading and deloading the viscous and sticky drug comprising a syringe body 1 having a lumen 2 therethrough characterized in that one end 3 of lumen being opposite to needle end 6 is provided with closing means 4, needle end 6 of lumen 2 is provided with closing means 7 comprising a glass bar 8 provided on its one side with a magnet bar 9, needle end 6 of lumen 2 and closing means 7 are covered with a cylindrical body 10 creating a cavity 11 around closing means 7, and cylindrical body 10 is provided on its end opposite to lumen 2 with a needle 12. In one embodiment, the present invention relates to An Auto-Destruct, Pre-loaded Syringe provided with actuator drive syringe.

### CROSS-REFERENCE TO RELATED APPLICATIONS

This application is a filing under 35 U.S.C. 371 of International Application No. PCT/IN2011/000821 filed Dec. 2, 2011, entitled “Auto-Destruct, Pre-Loaded Syringe,” which claims priority to Indian Patent Application No. 2903/DEL/2010 filed on Dec. 6, 2010, which applications are incorporated by reference herein in their entirety.

### FIELD OF THE INVENTION

The present invention relates to an auto-destruct, pre-loaded syringe, particularly it relates to auto-destruct, pre-loaded syringe for storage and delivery of viscous, sticky and chemically reactive drugs. More particularly, it relates to auto-destruct, pre-loaded syringe for storage and delivery of viscous, sticky and chemically reactive drugs, for example injectable contraceptives containing dimethyl sulfoxide [DMSO] required for injecting into lumen of vas deferens.

### BACKGROUND OF THE INVENTION

The vas deferens is a tubular structure linking the testes of the male to the ejaculatory duct. The scrotum of the male contains two testes and each testes has an independent vas deferens. Each vas deferens has a muscular wall and a central lumen which runs along the axis of the vas deferens. The spermatic fluid contains sperms flowing along lumen of the vas deferens being transported from the testes to the ejaculatory duct. In cross-section, the vas deferens has typically an outer diameter of about 2.5 mm or more and the lumen has a diameter of about 0.6 mm. The vas deferens is, therefore, a thick walled tube with very narrow lumen. The lumen will stretch when an injection needle is inserted into the lumen.

For the contraceptive application as well as for the diagnostic tests, there is a need to inject drugs into the lumen of the vas deferens. For this operation, the injection needle needs to be so handled that it penetrates the skin of the scrotum, penetrates the muscular wall of the vas deferens, and correctly and accurately enters the lumen of the vas deferens so that the desired dosage of the drug is delivered into the lumen without puncturing the walls of the vas deferens. This procedure is termed as “percutaneous injection” which implies injection through the skin without directly seeing the structure which in the present case is very narrow lumen of vas deferens.

The alternative to the percutaneous procedure is to cut open the skin with a scalpel, expose the vas deferens and inject the drug under direct visualization of the vas deferens. The “direct vision method” being a surgical procedure needs to be replaced by a percutaneous procedure.

The vas deferens is within the scrotal sac and is not visible. Being somewhat firmer than the skin tissue the vas can be felt by gripping the skin between two fingers but its diameter cannot be judged accurately because the skin thickness is about 2.5 mm or more.

Further, aligning the needle to be precisely over the central part of the vas deferens is subject to errors. Therefore, after the tip of the needle is made to penetrate the scrotal skin, the tip may deviate from the axis of the lumen of the vas deferens if the scrotal skin thickness and the diameter of the vas deferens are not known accurately. Under such circumstances, it is also possible that one may also miss the vas deferens completely. More commonly, the needle may also penetrate some part of the wall of the vas deferens, but may not enter the lumen. Further, if the needle does not align properly with the lumen, the needle tip may enter the lumen and then puncture through the opposite wall of the vas deferens.

The above problem is further enhanced due to very viscous and sticky nature of the injectable contraceptive which comprises dimethyl sulfoxide [DMSO] in higher amount as main ingredient of the formulation.

As the injectable contraceptive drug comprising DMSO is very viscous and sticky, it requires special art to load the syringe [the injection or drug delivery system]. Further due to its viscous and sticky nature, it also requires very high pressure for loading the syringe [the injection or drug delivery system]. Still furthermore due to its viscous and sticky nature, it also requires very high pressure for deloading the syringe [the injection or drug delivery system] for injecting the contraceptive drug into lumen of vas deferens.

Further, as diameter of lumen of vas deferens is very small, about 0.6 mm, the syringe [the injection or drug delivery system] is required to have needle of very small diameter at least less than about 0.6 mm, which further enhances the requirement of very high pressure for injecting very viscous and sticky contraceptive drug into lumen of vas deferens.

Accordingly, due to very viscous and sticky nature of the injectable contraceptive drug comprising DMSO, the surgeon needs to know special art to load the syringe, and needs to have capability to generate very high pressure required for loading the syringe and capability to generate very high pressure required for deloading the syringe for injecting the contraceptive drug into very narrow lumen of vas deferens.

It has been observed that the surgeons injecting injectable contraceptive drug comprising DMSO into very narrow lumen of vas deferens generally cannot load the syringe with viscous and sticky drug and also cannot deload the drug for injecting into very narrow lumen of vas deferens, particularly during the percutaneous injection wherein the drug is required to be injected into very narrow lumen of the vas deferens while properly adjusting and handling the injection needle in such a manner that it first penetrates the skin of the scrotum, then penetrates the muscular wall of the vas deferens, and then correctly and accurately enters the narrow lumen of the vas deferens so that the drug is delivered only into the lumen without puncturing the walls of the vas deferens, that is the surgeon is required to inject the viscous and sticky drug without directly seeing the structure which in the present case is lumen of vas deferens, that's too under very high pressure.

The U.S. Pat. No. 2,176,042A relates to a syringe comprising containers for preservation of biologically active substances under vacuum after treating such substances by freezing and dehydration under high vacuum to produce desiccated substance wherein the containers are provided with means by which water or other aqueous liquids can be readily introduced into the containers without destroying the vacuum to restore the desiccated substance.

The U.S. Pat. No. 3,040,743A relates to a hypodermic syringe in which the liquid to be injected is contained in an ampoule under pressure, and the syringe is suitable for military persons against nerve gases enabling to inject an anti-dote in his own body.

The U.S. Pat. No. 3,892,237 relates to injecting devices of the type comprising, on the one hand, a glass ampoule with a breakable tip containing a liquid to be injected and compressed gas, and, on the other hand, an hypodermic needle and communicating means between said needle and said glass ampoule.

The U.S. Pat. No. 5,193,907 relates to a process and apparatus for the mixing and direct emplacement of a bone cement formed from two components, one liquid and one powder, wherein the apparatus comprises a substantially cylindrical hollow body, a cover which can be attached to the said body, said cover having an aperture or hole coaxial with the longitudinal axis of the body and a plunger consisting of a stem which can move axially inside the said hole and a head which together with body bounds the chamber which aseptically houses the powder component of the cement, in which said body has at its base and projecting outwardly a hollow cylindrical needle which can be closed off by means of a sealing plug and in which the plunger which can move within the cylindrical cavity of the said body has a cavity forming the chamber housing the container of the liquid component of the cement, fitted with means for breaking the container which can be operated from outside the chamber itself and in which the chamber housing the powder component and the chamber housing the container of the liquid component are placed by means of a plurality of holes protected by a filter and located on the said head.

The German Patent No. DE 440629C (D5) relates to an injection device, wherein the injection needle c having covering h is broken through hit or bump from outside by using a magnetic power (see Figure Abb. 1). As per this patent, to enable that the fluid b passes through the needle c in the space f (Fig. Abb. 1), one must either apply pressure on the fluid through indifferent gas or it is necessary that the syringe s of the ampoule or the part t must be broken away. This patent also acknowledges that in the latter case, of course, atmospheric air gets into the medicinal liquid.

The PCT Publication No. WO 2001/047586 (D6) relates to a device for injecting liquids, and to a propulsion system for the injection device, said propulsion system comprising a container, a source of potential energy for propelling a fluid with sufficient pressure through an orifice to create a jet enabling subcutaneous or intracutaneous delivery of the fluid, wherein the source of potential energy comprises a first compressible substance at a first pressure P1 within the container and at least a second compressible substance at a second pressure P2 lower than P1, whereby said potential energy is substantially compression energy of said substances, said first substance being a liquid, solid, or other non-gaseous substance as defined at ambient temperature and pressure.

The above described devices fail to provide solution to the existing problems, i.e. these devices cannot withstand the pressure required for loading and deloading the viscous and sticky injectable materials, like contraceptive drug comprising DMSO, particularly when the drug has to be injected in very narrow lumen of vas deferens.

It has been further observed that presently available injection syringes made of glass material cannot withstand the pressure required for loading and deloading the viscous and sticky injectable contraceptive drug comprising DMSO, particularly when the drug has to be injected in very narrow lumen of vas deferens, more particularly when the drug has to be injected during the percutaneous injection under high pressure.

Accordingly, there is a need to have pre-loaded syringe loaded with viscous and sticky injectable contraceptive drug comprising DMSO and suitable to withstand the pressure required for loading and deloading the viscous and sticky injectable contraceptive drug comprising DMSO, particularly when the drug has to be injected into very narrow lumen of vas deferens, more particularly when the drug has to be injected during the percutaneous injection.

Further, the glass syringe available in the art are re-usable, which has high risk of communicable disease. Therefore, there is also a need to have a syringe which is not only pre-loaded but is also auto-destructable.

Another problem faced with the syringes of prior art is that the entry of atmospheric gases and moisture into its lumen space cannot be avoided. Therefore, there is also a need to have a syringe which will not allow entry of atmospheric gases and moisture into its lumen space.

Still another problem faced with the syringes of prior art is that the drug pre-loaded therein remains in flow and circulation within its lumen during its storage. Therefore, there is also a need to have a syringe wherein the pre-loaded drug will not flow or circulate within its lumen.

Yet another problem faced with the syringes of prior art is that when drug is to be filled in from an injection vial, the probability of contamination of the drug with glass particles formed during breakage of drug vial cannot be ruled-out. Therefore, there is also a need to have a syringe wherein the probability of contamination of the pre-loaded drug with glass particles is ruled-out or at least minimized.

Additional problem faced with the syringes of prior art is that the required amount of drug cannot be delivered as probability of volumetric error due to misreading of graduation bars cannot be ruled-out or at least minimized. Therefore, there is also a need to have a syringe wherein the probability of volumetric error due to misreading of graduation bars is ruled-out or at least minimized.

The syringes known in the prior art also suffer from the problems of shaking of needle particularly during delivery of viscous and sticky injectable contraceptive drug comprising DMSO under high pressure, which may also result in right through puncture of the vas deference. Therefore, there is also a need to have a syringe wherein the probability of shaking of needle particularly during delivery of viscous and sticky injectable contraceptive drug comprising DMSO under high pressure and probability of right through puncture of the vas deference is avoided or at least minimized.

Accordingly, there is a need to have auto-destruct and pre-loaded syringe which is loaded with viscous and sticky injectable contraceptive drug comprising DMSO and which is capable of overcoming above-described problems of prior art at least avoiding requirement of special technique and high pressure to load the syringe with the drug before injecting the drug into lumen of vas deferens.

### OBJECTS OF THE INVENTION

Main object of the present invention is to provide auto-destruct and pre-loaded syringe which is loaded with viscous and sticky injectable drug, for example with viscous and sticky injectable contraceptive drug comprising DMSO and which is capable of overcoming above-described problems of prior art at least avoiding requirements of special technique to load and de-load the syringe, and high pressure to load the syringe with the drug before injecting the drug into lumen of vas deferens, and high pressure to de-load the syringe for safe and comfortable injection of the drug into lumen of vas deferens, particularly when the drug has to be injected into very narrow lumen, for example in the lumen of vas deferens, more particularly when the drug has to be injected during the percutaneous injection.

Other objects and advantages of the present invention will be apparent from the following description of the present invention when read in conjunction with accompanying drawings, which are not intended to limit the scope of present invention.

It may be noted that the accompanying drawings are not to scale, and have been incorporated for the purpose to illustrate the present invention.

### BRIEF DESCRIPTION OF THE ACCOMPANYING FIGURES

FIG. 1 illustrates auto-destruct, pre-loaded syringe [injection or drug delivery system] in accordance with one of the preferred embodiments of the present invention.

FIG. 2 illustrates auto-destruct, pre-loaded syringe [injection or drug delivery system] during usage in accordance with one of the preferred embodiments of the present invention.

FIG. 3 illustrates auto-destruct, pre-loaded syringe [injection or drug delivery system] during usage to de-load the drug therefrom in accordance with one of the preferred embodiments of the present invention.

### DESCRIPTION AND PREFERRED EMBODIMENTS OF THE INVENTION

With aim to overcome above problems of the prior art, the inventor has found that if one end of the lumen of the syringe is provided with a closing means comprising a glass bar provided with a magnet bar on its one side, it surprisingly and unexpectedly, not only allows safe and comfortable loading and storage of the drug, but also allows its safe and comfortable delivery even under high pressure.

Accordingly, the present invention relates to auto-destruct, pre-loaded syringe comprising a syringe body 1 having a lumen 2 therethrough characterized in that
one end 3 of lumen being opposite to needle end 6 is provided with closing means 4,
needle end 6 of lumen 2 is provided with closing means 7 comprising a glass bar 8 provided on its one side with a magnet bar 9,
needle end 6 of lumen 2 and closing means 7 are covered with a cylindrical body 10 creating a cavity 11 around closing means 7, and
cylindrical body 10 is provided on its end opposite to lumen 2 with a needle 12.

In accordance with one of the preferred embodiments of the present invention the syringe is loaded with viscous and sticky injectable drug.

In accordance with one of the preferred embodiments of the present invention the viscous and sticky injectable drug is viscous and sticky injectable contraceptive drug comprising DMSO.

In accordance with one of the preferred embodiments of the present invention the end 3 of lumen 2 is preferably a conical structure provided with a closing means 4 consisting of a plugging means.

In accordance with one of the preferred embodiments of the present invention the end 3 having conical structure and provided with a plugging means 4 are preferably provided with additional closing cap 5 which has advantage of providing an air-tight sealing, which can be opened only when the pre-loaded drug is to be de-loaded.

In accordance with one of the preferred embodiments of the present invention the outer circumference of end 3 and inner circumference of additional closing cap 5 are provided with narrow spiral grooves which constitute screw like structure so that when closing cap 5 is fitted over the end 3, it is retained firmly and in-turn the plugging means 4 is retained, which has advantage of providing an air-tight sealing, which can be opened only when the drug is to be loaded before supply of syringe as pre-loaded syringe.

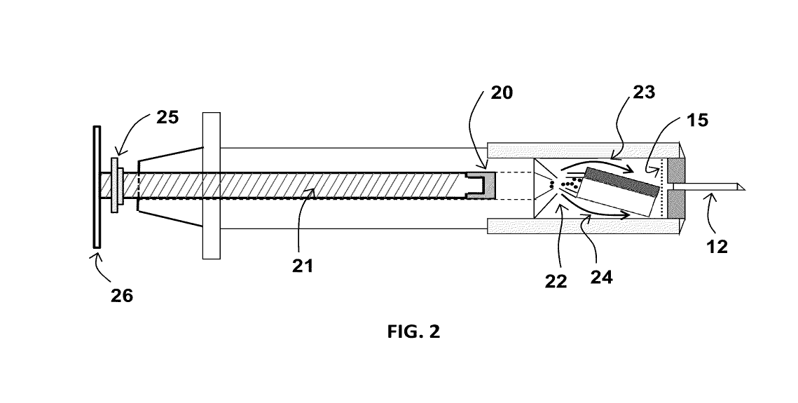

It has been observed that very minimal amount of environmental moisture and environmental gases, particularly oxygen, may leak into the lumen while closing of end 3 of the lumen after loading of syringe, which affects only that portion of the drug which is near the syringe flange 16 and this portion of the drug remains in the vicinity of the piston rubber cap 20 (re FIG. 2) and does not go into the body.

In accordance with one of the preferred embodiments of the present invention the magnet bar 9 is fixed onto the glass bar 8 with biocompatible adhesive, which is capable of fixing magnet bar onto glass bar.

In accordance with one of the preferred embodiments of the present invention the biocompatible adhesive is biocompatible cyanoacrylate adhesive.

In accordance with one of the preferred embodiments of the present invention the magnet bar 9 is in a sheet form which is fixed onto the glass bar 8 with the adhesive.

The closing means 7 comprising the glass bar 8 provided with magnet sheet 9 have been found to have surprising and unexpected advantage of opening lumen for release of drug therefrom for its delivery.

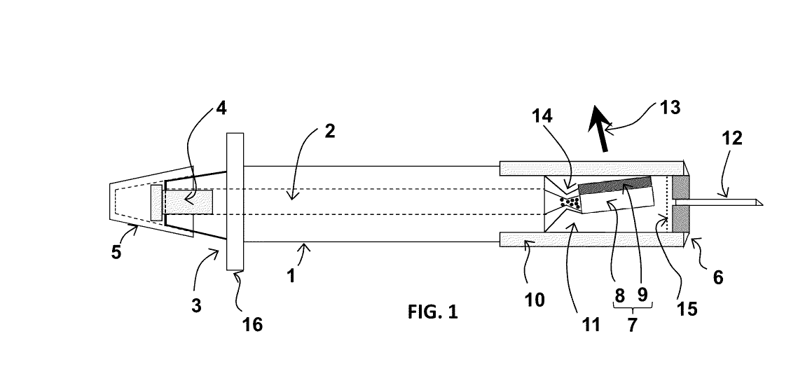

In accordance with one of the preferred embodiments of the present invention the magnet bar 9 is in a sheet form which is fixed onto the glass bar 8 with the adhesive so as to have its axis of magnetization, as shown with arrow 13 in FIG. 1, perpendicular to the long axis of the magnet bar 9.

In accordance with another preferred embodiment of the present invention, the closing means 7 comprising glass bar 8 and magnet bar 9 is tilted towards side of magnet bar 9. The perpendicular axis of magnetization of magnet bar 9 and tilted closing means 7 comprising the magnet bar have been found to have surprising and unexpected advantage of substantial enhancement of ease of opening of lumen by removing the closing means 7 for release of drug from the lumen for its delivery.

In accordance with one of the preferred embodiments of the present invention the closing means 7 comprising the glass bar 8 provided with magnet sheet 9 is fixed onto needle end of the syringe in a manner forming a notch 14 between the syringe body 1 and closing means 7 which has been surprisingly found to have advantage of forming a weak point. It has been observed that under the influence of the rotational torque this notch breaks first and makes way for release of drug.

In accordance with one of the preferred embodiments of the present invention, the closing means 7 is only fixed onto the needle end of the syringe body and free on its opposite end, which allows its easy release on breakage of notch 14.

As the notch 14 is made of glass material used in manufacture of syringe body, the probability of glass particles getting into drug released cannot be avoided. The present invention overcomes this problem by providing a coating on the outer surface of the notch.

In accordance with one of the preferred embodiments of the present invention the coating on the notch comprises styrene maleic anhydride (SMA).

In accordance with one of the preferred embodiments of the present invention the coating is prepared by dissolving styrene maleic anhydride (SMA) in a solvent and then evaporating the solvent to result in plastic coating.

In accordance with one of the preferred embodiments of the present invention the solvent is preferably 1,2 dichloro methane.

It has been found that the thin plastic coating of present invention on notch surprisingly and unexpectedly prevents glass particle formed on breakage being released in the drug.

The present invention further rules out or at least minimizes any probability of glass particles getting mixed into the drug by additionally applying a small amount of SMA powder in the form of compact around the lumen wall of the notch.

In accordance with one of the preferred embodiments of the present invention the compact of SMA powder may be applied at the time of loading the syringe.

It has been observed that on contact with the drug in the cavity 11, the SMA powder converts to a thick gel, which traps the glass particles, if any released inside the cavity on breakage of the notch.

Further, in accordance with one of the preferred embodiments of the present invention, the thickness of the glass used to manufacture the notch is very small which further reduces probability of glass particles being released at the time of breakage.

Further, in accordance with one of the preferred embodiments of the present invention, the needle end of the cylindrical body 10 is provided with a filtering means 15 which ensures that neither the coating nor the gel of SMA nor the glass particles get into the drug being released through the needle.

The closing means 7 and cylindrical body 10 have additional advantage of avoiding entry of atmospheric gases and moisture into the lumen space.

The closing means 4 and closing means 7 have advantage of avoiding flow and circulation of drug within the lumen during the storage and supply of pre-loaded syringe, and provide functional protection to drug during storage.

As soon as the closing means 7 is released, the syringe gets destroyed permanently and cannot be re-used. Therefore, the present invention provides a pre-loaded syringe, which is also auto-destruct syringe.

Accordingly, the present invention overcomes above described problems of the prior art by providing a pre-loaded syringe having a closing means 7 provided at the needle end of the syringe body.

In another embodiment, the present invention overcomes above described problems of the prior art by providing a pre-loaded syringe having a closing means 7 provided at the needle end of the syringe body and fixed onto the syringe body by forming a notch 14.

Accordingly, in one embodiment, the present invention relates to auto-destruct, pre-loaded syringe comprising a syringe body 1 having a lumen 2 therethrough characterized in that
one end 3 of lumen being opposite to needle end 6 is provided with closing means 4,
needle end 6 of lumen 2 is provided with closing means 7 comprising a glass bar 8 provided on its one side with a magnet bar 9,
closing means 7 is connected to syringe body 1 by forming a notch 14,
needle end 6 of lumen 2 and closing means 7 are covered with a cylindrical body 10 creating a cavity 11 around closing means 7,
cylindrical body 10 is provided on its end opposite to lumen 2 with a needle 12.

In another embodiment, the present invention overcomes above described problems of the prior art by providing a pre-loaded syringe having a closing means 7 provided at the needle end of the syringe body and fixed onto the syringe body by forming a notch 14, wherein the notch 14 is provided with a coating.

In another embodiment, the present invention overcomes above described problems of the prior art by providing a pre-loaded syringe having a closing means 7 provided at the needle end of the syringe body and fixed onto the syringe body by forming a notch 14, wherein the notch 14 is provided with one or more of the coating, compact of SMA powder.

In another embodiment, the present invention overcomes above described problems of the prior art by providing a pre-loaded syringe having a closing means 7 provided at the needle end of the syringe body and fixed onto the syringe body by forming a notch 14, wherein the notch 14 is provided with one or more of the coating, compact of SMA powder, and the glass used to manufacture notch is of very low thickness.

The present syringe when to be supplied by the manufacturer will be in the “pre-loaded” form [FIG. 1], wherein the drug is filled in the lumen of the syringe, and the lumen, after filling of drug is also sealed on its end 3. The closing means 4 and 7 on both ends of the syringe body ensure that there is no entry of atmospheric gases and moisture into the lumen space, and there is no flow or circulation of the drug within the lumen during its storage and transportation. To release the drug from the syringe, an external magnet [not shown in figures], which is preferably high pole strength external magnet, is brought close to the closing means 7 in such a manner that it is brought close to the vicinity of the bar magnet 9. It is not be necessary to make physical contact between the syringe and the external magnet. The axis of magnetization of the external magnet is preferably kept parallel to the axis of magnetization of the bar magnet 9, but in such an orientation that there is a repulsive force on the bar magnet and in consequence on the glass bar 8. The glass bar 8 having a fixation on one end [end towards notch 14] and being free on the other [opposite] end is subjected to a rotational torque, which results in breakage of notch 14 which serves as a “weak point”. The external magnet can, thereafter, be removed.

Following the breakage at the notch 14, the closing cap 5 is removed preferably by a rotation and pull, and the plugging means 4 is pulled out and the piston rod 21 provided with the piston cap 20 is inserted into the lumen of syringe [FIG. 2]. As the piston is advanced into the syringe barrel, the drug starts flowing from the point of broken notch 22 along the path as shown by arrows 23 and 24. As thickness of glass at the notch is very small the probability of glass particles being released at the time of breakage is very low. The possibility of glass particles getting into drug is further reduced due to the coating of styrene maleic anhydride (SMA) on the notch area of the syringe.

In accordance with one of the preferred embodiments of the present invention, the SMA is dissolved in a solvent, preferably in 1,2 dichloro methane and coating is formed after the solvent is evaporated. The resulted thin plastic coating has been found capable of preventing formation of glass particle on breakage of notch.

Further, additionally in the lumen on the two sides of the notch a small amount of SMA powder is compacted at the time of loading the syringe. The drug on contact with the powder becomes a thick gel, and any glass particle released inside the barrel gets trapped in the gel.

Further, additionally, the drug after release from the point of broken notch is filtered by passing through a filter means 15.

In accordance with one of the preferred embodiment of the present invention, the filtering means 15 is a perforated stainless steel sheet. It has been found that glass particles, if any, get entrapped into or with coating, and with or into the gel formed from compact of SMA powder, and only the drug free from any contamination flows into through the filtering means 15 and the needle 12.

A predetermined specific volume of the drug is required to be injected into each subject. In the surgical room often volumetric errors in injection occur because in graduated syringes the graduation bars are misread. In the present invention this problem is overcome by providing a judiciously designed piston rod 21 provided with threads like structure thereon and volume adjustable means 25. After the doctor has pushed the piston to the extent that the drug just begins to come out of the needle point the volume adjustable means 25 is adjusted to such a distance from the glass cone that when the injection is being done only the desired volume of the drug is delivered which is achieved as soon as volume adjustable means abuts against the glass cone.

Therefore, the syringe of present invention is capable of delivering only the correct volume even without observing graduations during the injection process, and hence, the possibility of error is eliminated.

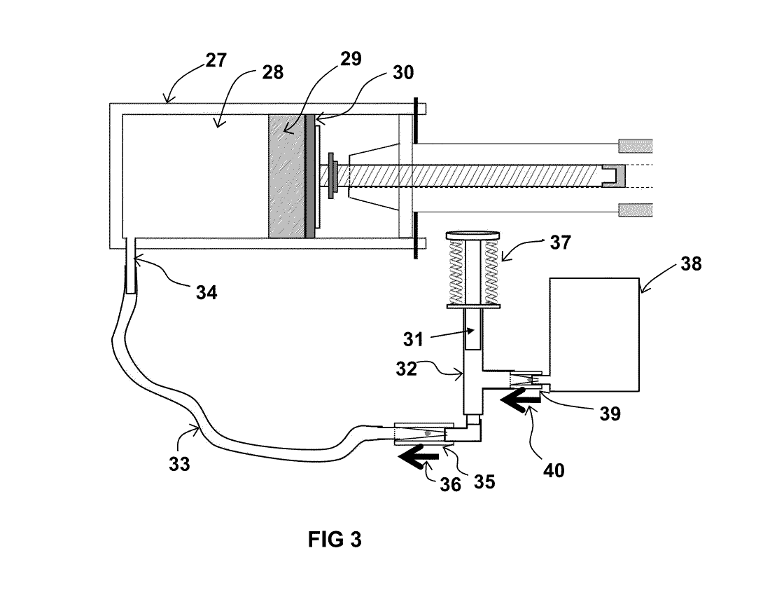

In accordance with preferred embodiment of the present invention, the drug is viscous drug. It has been observed that when a viscous drug has to be injected through a narrow bore needle the force required on the piston rod is high. With the doctor having two fingers on the flange and thumb on the thumb rest 26 of piston 21, the grip force exertion leads to tremor of the hand of the doctor. In the consequences, the needle shakes and the probability of puncturing right through the vessel is substantially increased. The present invention overcomes this hazardous problem by judiciously providing piston drive mechanism [FIG. 3] comprising an actuator 27, which is preferably cylindrical actuator, having a cavity 28 and a piston means which is a combination of a rubber piston 29 of cylinder and a thrust means 30, which is preferably a metallic thrust means and positioned in a manner that the thrust means 30 abuts against the thumb rest means 26 of the piston. The actuator cylinder 27 is filled by means of movement of actuator drive syringe piston 31. This mechanism is provided with an actuator drive syringe 32 to pump liquid such as water via the flexible connecting means 33 and the inlet to cylinder 34. When the piston 31 of actuator drive syringe 32 is pressed into the actuator drive syringe, the liquid in this syringe flows through the one way valve means 35 in the direction of flow 36 through the flexible connecting means into the cylinder cavity 28. The rubber piston 29 of cylinder and thrust means 30 are pushed forward so that the thumb rest 26 of piston and in turn the piston rod 21 are pushed forward and drug is expelled via the needle [FIG. 2]. The extent of movement is proportional to the volume of liquid pumped by one stroke of the piston of actuator drive syringe. At the end of the stroke of the piston of actuator drive syringe, this piston is released. The compression means 37 automatically pushes the piston 31 of actuator drive syringe 32 to increase the volume space in the actuator drive syringe. A low pressure is generated within this syringe and liquid flows from the water reservoir means 38 into the actuator drive syringe via the one way valve 39 in the direction of flow 40. On repeating the cycle, the piston rod is pushed forward in steps. The stepped infusion has a number of advantages over continuous infusion. The piston of actuator drive syringe can be cyclically moved by an assistant to the doctor or the doctor can operate the piston of actuator drive syringe with his own foot. Hence there is no stress on the doctor's hand and probability of accidental counter puncture during injection is avoided.

1. Auto-destruct, pre-loaded syringe comprising a syringe body (1) having a lumen (2) therethrough wherein
one end (3) of lumen being opposite to needle end (6) is provided with closing means (4),
characterized in that
needle end (6) of lumen (2) is provided with closing means (7),
said closing means (7) comprises a glass bar (8) provided on its one side with a magnet bar (9),
wherein said closing means (7) comprising glass bar (8) and magnet sheet (9) is fixed onto said needle end (6) of the syringe forming a notch (14) between said syringe body (1) and said closing means (7),
wherein said notch (14) is provided with a coating,
wherein said coating comprises styrene maleic anhydride (SMA),
needle end (6) of lumen 2 and dosing means (7) are covered with a cylindrical body (10) creating a cavity (11) around closing means (7), and
cylindrical body (10) is provided on its end opposite to lumen (2) with a needle (12).

2. The auto-destruct, pre-loaded syringe as claimed in claim 1, wherein the syringe is loaded with viscous and sticky injectable drug.

3. The auto-destruct, pre-loaded syringe as claimed in claim 2, wherein the injectable drug is viscous and sticky injectable contraceptive drug comprising dimethyl sulfoxide.

4. The auto-destruct, pre-loaded syringe as claimed in claim 1, wherein the end (3) has conical structure and is preferably provided with additional closing cap (5).

5. The auto-destruct, pre-loaded syringe as claimed in claim 1, wherein the outer circumference of end (3) and inner circumference of additional closing cap (5) are provided with narrow spiral grooves.

6. The auto-destruct, pre-loaded syringe as claimed in claim 1, wherein the magnet bar (9) is fixed onto the glass bar (8) with biocompatible adhesive.

7. The auto-destruct, pre-loaded syringe as claimed in claim 6, wherein the biocompatible adhesive is biocompatible cyanoacrylate adhesive.

8. The auto-destruct, pre-loaded syringe as claimed in claim 1, wherein the magnet bar (9) is in a sheet form.

9. The auto-destruct, pre-loaded syringe as claimed in claim 1, wherein the magnet bar (9) is fixed onto the glass bar (8) so as to have its axis of magnetization perpendicular to the long axis of the magnet bar (9).

10. The auto-destruct, preloaded syringe as claimed in claim 1, wherein the closing means (7) comprising glass bar (8) and magnet bar (9) is tilted towards side of magnet bar (9).

11. The auto-destruct, pre-loaded syringe as claimed in claim 1, wherein the closing means (7) is fixed onto the needle end of the lumen of the syringe body and free on its opposite end.

12. The auto-destruct, pre-loaded syringe as claimed in claim 1, wherein said coating is plastic coating.

13. The auto-destruct, pre-loaded syringe as claimed in claim 1, wherein additionally SMA powder is applied in the form of compact around the lumen wall of said notch (14).

14. The auto-destruct, pre-loaded syringe as claimed in claim 1, wherein the needle end of the cylindrical body (10) is provided with a filtering means (15).

15. The auto-destruct, pre-loaded syringe as claimed in claim 14, wherein the filtering means (15) is a perforated stainless steel sheet.

16. The auto-destruct, pre-loaded syringe as claimed in claim 1, wherein the syringe is provided with piston drive mechanism comprising an actuator (27) having a cavity (28) and a piston means consisting of rubber piston (29) of cylinder and a thrust means (30) positioned in a manner that the thrust means (30) abuts against the thumb rest means (26) of the piston.

17. The auto-destruct, pre-loaded syringe as claimed in claim 16, wherein the actuator cylinder (27) is filled by means of movement of actuator drive syringe piston (31).

18. The auto destruct, pre-loaded syringe as claimed in claim 16, wherein the piston drive mechanism is provided with an actuator drive syringe (32).

19. The auto-destruct, pre-loaded syringe as claimed in claim 18, wherein the actuator drive syringe (32) is provided with a piston (31).

20. The auto-destruct, pre-loaded syringe as claimed in claim 19, wherein the piston (31) is connected to a compression means (37).

21-34. (canceled)
